(19) United States
(12) Patent Application Publication
(10) Pub. No.: US 2023/0233057 A1
(43) Pub. Date: Jul. 27, 2023

(54) Visual data transfer between the end and side of a multimode fiber

(71) Applicant: RAMOT AT TEL-AVIV UNIVERSITY LTD., Tel Aviv (IL)

(72) Inventors: Avishay Eyal, Givat Shmuel (IL); Alon Bahabad, Herzliya (IL); Daniel Marima, Tel Aviv (IL); Barak Hadad, Petah Tikva (IL); Sahar Froim, Rishon Lezion (IL)

(21) Appl. No.: 18/011,203

(22) PCT Filed: Jul. 18, 2021
(86) PCT No.: PCT/IB2021/056475

§ 371 Date: Dec. 19, 2022

Related U.S. Application Data
(60) Provisional application No. 63/058,727, filed on Jul. 30, 2020.

(51) Int. Cl. A61B 1/00

(52) U.S. Cl. CPC A61B 1/00009; A61B 1/00167; G02B 6/0288

(57) ABSTRACT

A method for transmitting information includes deriving a transfer function that relates a first image formed over a first area on an end face (38) of a multimode optical fiber (40) and a second image formed over a second area extending over a side (42) of the multimode optical fiber. Optical information is input to the multimode optical fiber through one of the first and second areas. Following transmission of the optical information through the multimode optical fiber, the optical information that is output from the other of the first and second areas is detected and decoded using the transfer function.

### CROSS-REFERENCE TO RELATED APPLICATION

This application claims the benefit of U.S. Provisional Patent Application 63/058,727, filed Jul. 30, 2020, which is incorporated herein by reference.

### FIELD OF THE INVENTION

The present invention relates generally to optical methods and devices, and particularly to transmission of optical images through multimode fibers.

### BACKGROUND

Light propagation in a medium with random inhomogeneities is generally accompanied by some degree of scattering. If the characteristic size of the inhomogeneities is much smaller than the wavelength of the light, the scattering is called Rayleigh scattering. The interference pattern of the randomly scattered light forms a granular image known as speckles. (The terms “light” and “optical radiation” are used in the context of the present description and in the claims to refer to electromagnetic radiation in any of the visible, infrared, and ultraviolet spectral ranges.)

Rayleigh scattering in optical fibers has been studied extensively. Most studies, however, pertained to scattered light that remained confined in the fiber and propagated either forward or backward. Considerably less attention has been given to the light that is scattered sideways outside the fiber. In multimode fibers, the granular speckle pattern that side-scattered light produces makes it difficult to retrieve spatial information carried by the light.

### SUMMARY

Embodiments of the present invention that are described herein provide improved methods and apparatus for transmission of information through optical fibers.

There is therefore provided, in accordance with an embodiment of the invention, a method for transmitting information, which includes deriving a transfer function that relates a first image formed over a first area on an end face of a multimode optical fiber and a second image formed over a second area extending over a side of the multimode optical fiber. Optical information is input to the multimode optical fiber through one of the first and second areas. Following transmission of the optical information through the multimode optical fiber, the optical information that is output from the other of the first and second areas is detected. The detected optical information is decoded using the transfer function.

In a disclosed embodiment, the second image includes a speckle pattern formed by Rayleigh scattering of coherent light within the optical fiber.

In some embodiments, deriving the transfer function includes projecting multiple input images onto the first area, capturing respective output images of the second area responsively to the input images, and optimizing the transfer function so as to transform the input images into the respective output images. In one embodiment, optimizing the transfer function includes training a neural network to relate the respective output images to the input images.

Additionally or alternatively, deriving the transfer function includes finding multiple, respective transfer function components relating the first image formed over the first area on the end face of the multimode optical fiber to multiple second areas extending in different, respective locations over the side of the multimode optical fiber.

Further additionally or alternatively, deriving the transfer function includes generating the transfer function so as to decode the detected optical information regardless of changes in the transmission due to variations in a shape of the multimode optical fiber.

In some embodiments, inputting the optical information includes applying a pattern of light to the end face of the multimode optical fiber, and decoding the detected optical information includes reconstructing the pattern by applying the transfer function to the light emitted from the side of the multimode optical fiber.

In other embodiments, inputting the optical information includes receiving light through the side of the multimode optical fiber, and decoding the detected optical information includes outputting an image of an area radial to the multimode optical fiber responsively to the light received through the side of the multimode optical fiber.

Typically, at least the second area of the side of the optical fiber is jacketless.

There is also provided, in accordance with an embodiment of the invention, apparatus for transmitting information, including a multimode optical fiber, which includes a first area on an end face of the multimode optical fiber and a second area extending over a side of the multimode optical fiber. An optical input assembly is configured to input optical information to the multimode optical fiber through one of the first and second areas. A detector is configured to detect the optical information that is output from the other of the first and second areas. A processor is configured to derive a transfer function that relates a first image formed over the first area on the end face of a multimode optical fiber and a second image formed over the second area extending over the side of the multimode optical fiber, to receive the optical information detected by the detector, and to decode the detected optical information using the transfer function. The present invention will be more fully understood from the following detailed description of the embodiments thereof, taken together with the drawings in which:

### BRIEF DESCRIPTION OF THE DRAWINGS

FIG. 1 is a schematic side view of a system for transmission of information through an optical fiber, in accordance with an embodiment of the invention;

FIGS. 2 and 3 are schematic representations of images that were input to and output from the system of FIG. 1, illustrating methods for transmitting information through the optical fiber, in accordance with embodiments of the invention; and

FIG. 4 is a schematic pictorial illustration of an endoscopic imaging apparatus, in accordance with an embodiment of the invention.

### DETAILED DESCRIPTION

### Overview

Although most of the light in an optical fiber is guided through the fiber core, there is always some small amount of the light that is scattered sideways due to Rayleigh scattering from inhomogeneities in the fiber core. Thus, a small fraction of the guided light escapes out through the side of the fiber. For coherent light propagating in a multimode optical fiber (referred to hereinafter simply as a multimode fiber, or MMF), the random image formed by side-scattered light at any location along the fiber is a granular speckle pattern, as is commonly observed in random coherent scattering. This speckle pattern makes it difficult to retrieve spatial information carried by the side-scattered light. The ability to efficiently analyze these speckle patterns, and thus gather seemingly lost information from these patterns, has many potential applications.

Embodiments of the present invention that are described herein provide techniques for associating a first image at the end face of an optical fiber with a second image formed over an area of the side of the fiber. For example, in some embodiments an input image at the end face is associated with a corresponding output image of a speckle pattern formed by Rayleigh scattering to the sides of the fiber. On this basis, a transformation is derived between the image information that is input to the end face of fiber and the speckle pattern detected at locations along the side of the fiber. Further embodiments apply the transformation in the inverse direction, for visual sensing of the surroundings at the sides of the fiber.

In the disclosed embodiments, a jacketless fiber is used to allow the sideways-scattered light to reach the surrounding of the fiber. Alternatively, the jacket may be removed only in the specific areas of the side through which information is to be received or transmitted.

Thus, in the disclosed embodiments, a processor derives a transfer function relating an image formed over a first area on an end face of a MMF and a corresponding image formed over a second area extending along a side of the MMF. Optical information can then be input to the MMF through one of the first and second areas, and will be output from the other of the areas following transmission of the optical information through the MMF. An image sensor detects this output optical information, and the processor decodes the detected optical information using the transfer function. In this manner, for example, an image input to the end face of the MMF can be output through the side of the MMF and there detected and decoded.

Alternatively or additionally, an image can be input through the side of the MMF and then detected and decoded at the end face. In this case, light is captured through the side of the MMF, and the processor can decode the optical information detected at the end face in order to form and output an image of an area radial to the multimode optical fiber. The image that is input through the side of the MMF can be illuminated by light originally input through an end-facet, scattered outside the fiber, back-reflected from objects in the vicinity of the fiber and scattered back into the fiber core, and finally detected at the same end facet of the fiber. This sort of application is useful, for example, in endoscopic imaging.

In some embodiments, in order to derive the transfer function, multiple input images are projected onto the area of the end face, and respective output images are captured of the area of interest on the side of the fiber. The transfer function is optimized so as to transform the input images into the respective output images. For this purpose, for example, a neural network may be trained to relate the respective output images to the input images.

In some embodiments, the transfer function derivation is extended to find multiple, respective transfer function components relating an image formed over the area of the end face to multiple different areas extending in different, respective locations over the side of the multimode optical fiber.

Additionally or alternatively, the transfer function is derived so as to decode the detected optical information regardless of changes in the transmission of light through the fiber that occur due to variations in the shape of the multimode optical fiber.

### System Description

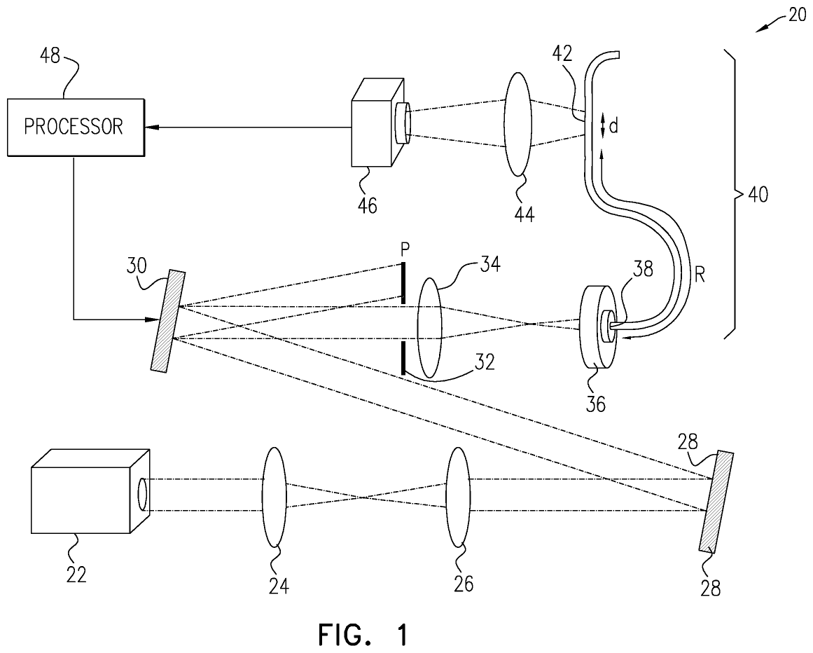

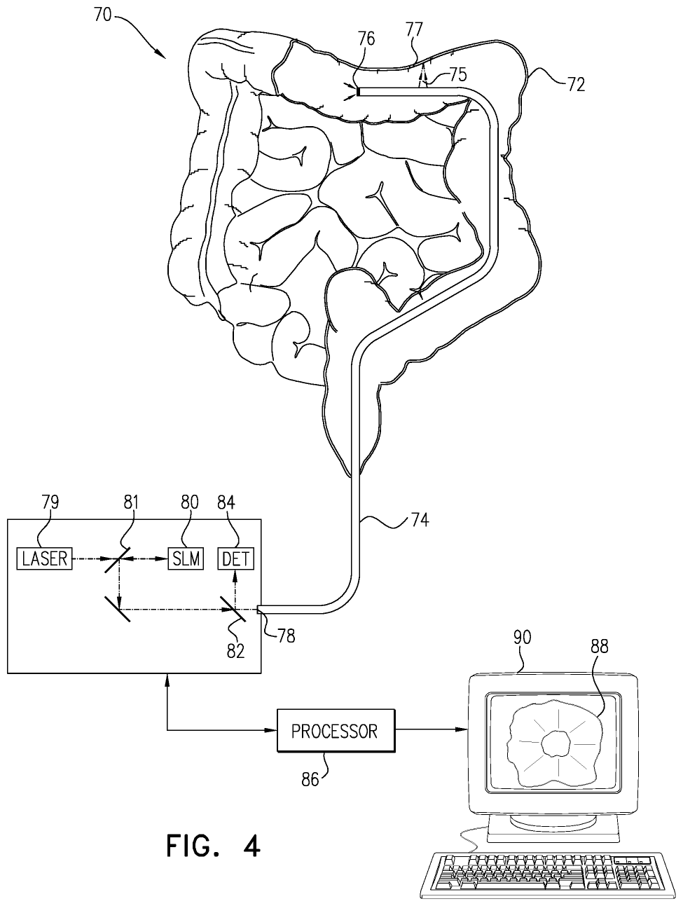

FIG. 1 is a schematic side view of a system 20 for transmission of information through a multimode optical fiber 40, in accordance with an embodiment of the invention. This figure illustrates a particular experimental setup that can be used to transmit images through an end face 38 of fiber and then to receive and process images of the resulting speckle pattern that is emitted through an area of a side 42 of the fiber. Alternatively, other means for image formation, transmission, and detection, as are known in the art, may be used for transmission of image information through an MMF such as fiber 40. Furthermore, image information may be transmitted through the fiber in the opposite direction, from side 42 to end face 38, as illustrated, for example, by the embodiment of FIG. 4.

In the embodiment of FIG. 1, a laser 22 emits a coherent beam of light, which is directed by beam-expanding optics, including lenses 24 and 26 and a mirror 28, toward a spatial light modulator (SLM) 30. SLM 30 comprises a phase modulation array, for example a liquid crystal pixel array, such as the PLUTO spatial light modulator produced by HOLOEYE Photonics AG (Berlin, Germany). Alternatively, other light modulation devices may be used, such as a deformable mirror, a digital micro-mirror array, or a passive diffractive element.

Processor 48 controls SLM 30 to modulate a desired spatial phase or amplitude pattern onto the laser beam. The first diffraction order from the SLM passes through a pinhole 32 and is then focused by a lens 34 through a fiber coupler 36, which enables precise alignment of the image of the modulated pattern onto end face 38. Lens 34 and the optics inside fiber coupler 36 may advantageously be configured for this purpose as a 4f system. In the present embodiment, fiber 40 comprises a bare MMF, for example a two-meter length of FG105LCA fiber, produced by Thorlabs Inc. (Newton, N.J.). Laser 22, SLM 30, and the passive optical elements described above together constitute the optical input assembly of system 20. Other sorts of optical input assemblies can alternatively be used to modulate optical information onto end face 38, as will be apparent to those skilled in the art after reading the present description, and are likewise considered to be within the scope of the present invention.

An imaging lens 44 collects and focuses light emitted from an area of length d along side 42 into a camera 46, which comprises a suitable detector, such as a CCD or CMOS image sensor. The area from which the side-emitted light is collected is located at a distance R from end face 38.

Processor 48 receives and analyzes the images captured by camera 46. The processor retrieves the amplitude and/or the phase of the light that was input through end face 38 of fiber 40 based on the speckle pattern formed at side 42 of the fiber, for example using a convolutional neural network (CNN). The CNN is initially trained, as described further hereinbelow, using a training set of known input images, together with the corresponding speckle pattern output images captured by camera 46. Thus, the CNN effectively learns the transfer function between the end face and sides of fiber 40. The CNN can be trained using speckle patterns taken from multiple locations along side 42 of fiber 40, as well as using speckle patterns captured under different conditions of curvature of the fiber. Alternatively, other machine learning and optimization techniques can be used in extracting the transfer function of the fiber.

Following the training stage, processor 48 can apply the CNN to decode unknown speckle patterns received through side 42 of fiber 40, and thus to reconstruct image information that has been focused onto end face 38. In some embodiments, these capabilities are applied in distributed optical imaging systems and unique optical links that can be interfaced via the sides of the fiber.

System 20 may be operated so as to extract the respective components of the transfer function for multiple specific points along the length of side 42. This transfer function will then indicate to processor 48 the amplitude and/or phase pattern that should be applied by SLM 30 to end face 38 in order to focus light at each of these specific points. The SLM setting can be modulated over time among these patterns in order to scan the focal point along side 42. Processor 38 may register the light that is scattered back into fiber 40 (either by reflection at the same frequency or fluorescence at another frequency) in order to build an image of the surroundings of the fiber.

As noted earlier, processor 48 may derive and apply the transfer function of fiber 40 for a specific location or multiple locations along side 42 in a way that is robust against bending of the fiber, for example by generalizing the training procedure described above to include different deformations of the fiber. Additionally or alternatively, a CNN may be trained to recognize the salient features carried by any specific information input into the fiber regardless of the position or deformation of the fiber, for example by searching for correlations and other statistical characteristics of the speckle patterns that are independent of the exact location along the fiber and/or its deformation.

As another option, a reference defect can be created, for example by direct laser writing, at the distal end facet of fiber 40. Reflection from this defect can then be used to extract the shape of the fiber - for example using machine learning methods. This specific information regarding deformation of the fiber can be used together with the images captured by camera 46 of side 42 to decode the input to end face 38 regardless of the shape of the fiber.

As another option, to enhance the efficiency of light scattering from within fiber 40 to side 42, optical scattering structures can be formed in desired locations within the fiber, for example by direct laser writing on either the core or cladding of the fiber. These scattering structures may be either ordered (such as Bragg gratings) or disordered.

### Proof of Concept

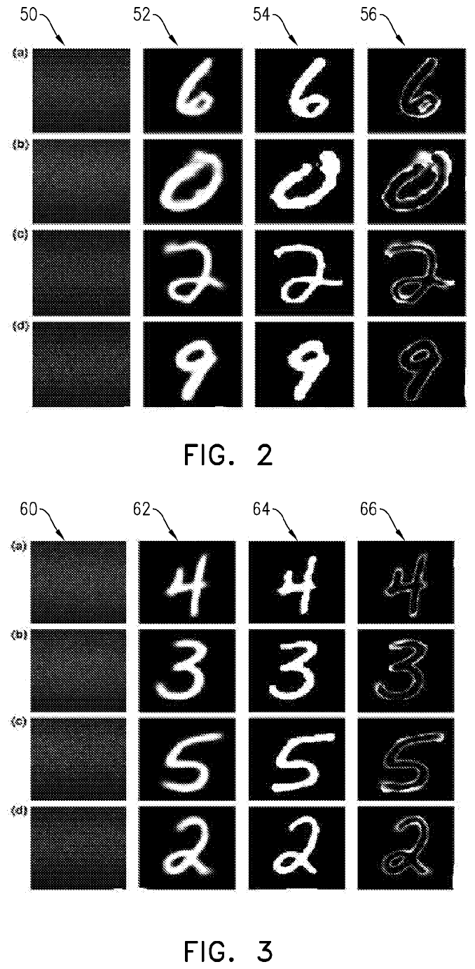

FIGS. 2 and 3 are schematic representations of images that were input to and output from system 20 (FIG. 1), illustrating methods for transmitting information through optical fiber 40, in accordance with embodiments of the invention. FIG. 2 shows the results of training a CNN to be applied by processor 48 to process speckle patterns output from a single location along side 42, whereas FIG. 3 shows the results of training over multiple locations. To produce these results, camera 46 captured images of an area of side 42 with d˜300 μm. The images contained the entire width of the fiber (which was ˜250 μm) . Each of the speckle images was normalized by its maximum value and then fed to the CNN. The CNN was defined as an encoder-decoder network, similar to that described by Li et al. in “Deep speckle correlation: A deep learning approach toward scalable imaging through scattering media,” published in Optica 5:10 (2018), pages 1181-1190, and incorporated herein by reference. Alternatively, other neural network architectures, as are known in the art, may be applied for these purposes.

Specifically, in FIG. 2, rows (a) and (b) show results obtained when the training and speckle output took place at a location on side 42 in proximity to end face 38 (i.e., with a small value of R), while rows (c) and (d) show the results at a location far form end face 38 (large R). The results show that the present techniques are largely independent of the length of fiber 40. In each row, a first column 50 shows the speckle image captured by camera 46 for a given image input at end face 38, while a second column 52 shows the image reconstructed by processor 48 using the trained CNN. The ground truth image, i.e., the image that was actually projected from SLM 30 onto end face 38, is shown in a third column 54, and the error between the reconstructed and ground truth images is shown in a fourth column 56. The pictured results were obtained using phase modulation in projecting the input images onto end face 38, but in other experiments, similar results were obtained using amplitude modulation.

In FIG. 3, the CNN was trained using speckle patterns obtained at four different locations, which were spaced 635 μm apart along the length of side 42. Each row shows the experimental results for a respective one of the four locations. Columns 60, 62, 64 and 66 have the same, respective meanings as the corresponding columns 50, 52, 54 and 56 in FIG. 2.

As can be seen in both of FIGS. 2 and 3, there was a close resemblance between the reconstructed and ground truth results, with only small error. These results thus demonstrate the ability of system 20 to recover information input to end face 38 of fiber 40 by learning and applying an appropriate transfer function to the speckle pattern emitted through side 42.

The slight blurring of the reconstructed images can be attributed to two factors: The first is the finite spatial bandwidth response of the optical components in the system, including the finite numerical aperture of fiber 40. The second factor is the neural network itself, since it is mainly composed of convolution operations, which may cause low-pass filtering.

Thus, FIGS. 2 and 3 illustrate the ability of system 20, with an appropriately-trained CNN, to analyze the speckle pattern formed at side 42 of a bare MMF due to random Rayleigh scattering, and to retrieve either the amplitude or the phase pattern of the light input to end face 38 of the fiber. The performance of the disclosed technique was found to be independent of the position of the fiber section being imaged (at least for the length scale of a few meters that was tested). FIG. 3 specifically shows that a single CNN can perform this task for speckle patterns taken from a predefined set of several different locations along the fiber. Although relatively simple images were used in the present experiments, the same principles can be applied in training a CNN to decode more complicated phase and amplitude patterns, thus increasing the amount of data that can be delivered sideways through the MMF.

The principles of the embodiment of FIG. 1 can be applied, inter alia, in developing a distributed transmitter network based on a single fiber and optical detectors with no additional optical components.

### Example Application in Endoscopic Imaging

FIG. 4 is a schematic pictorial illustration of an endoscopic imaging apparatus 70, in accordance with an embodiment of the invention. In apparatus 70, a multimode fiber 74 is inserted into a cavity 72, such as the intestine in a living body. In the pictured example, a laser 79 directs a beam of coherent radiation through a beamsplitter toward an SLM 80, which applies an appropriate amplitude and/or phase modulation to the beam. The modulated beam passes through another beamsplitter 82 and enters an end-face 78 of fiber 74. Fiber 74 captures image information by transmitting and receiving light through locations along the sides of fiber 74, for example in a beam 75 directed toward structures 77 in the area radially surrounding the fiber. In addition, fiber 74 may capture image information axially through a distal end 76 of the fiber.

For the purposes of apparatus 70, a processor 86 learns the transfer function of fiber 74 and applies the transfer function in focusing light to specific locations at the sides of the fiber. Based on this transfer function, processor 86 drives SLM 80 to generate images on an end face 78 of fiber 74, wherein each such image causes light to be scattered out of a different, known point on the side of the fiber, as illustrated by beam 75. The light that is scattered back from the tissue in cavity 72 (at the same wavelength as the transmitted light or at different wavelengths due to fluorescence) passes back through fiber 74 and is imaged via beamsplitter 82 onto a detector 84. Processor 86 receives and processes the output of detector 84, for example to reconstruct and display an image 88 of the interior of cavity 72 on a display screen 90.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for transmitting information, comprising:
deriving a transfer function that relates a first image formed over a first area on an end face of a multimode optical fiber and a second image formed over a second area extending over a side of the multimode optical fiber;
inputting optical information to the multimode optical fiber through one of the first and second areas;
following transmission of the optical information through the multimode optical fiber, detecting the optical information that is output from the other of the first and second areas; and
decoding the detected optical information using the transfer function.

2. The method according to claim 1, wherein the second image comprises a speckle pattern formed by Rayleigh scattering of coherent light within the optical fiber.

3. The method according to claim 1, wherein deriving the transfer function comprises projecting multiple input images onto the first area, capturing respective output images of the second area responsively to the input images, and optimizing the transfer function so as to transform the input images into the respective output images.

4. The method according to claim 3, wherein optimizing the transfer function comprises training a neural network to relate the respective output images to the input images.

5. The method according to claim 1, wherein deriving the transfer function comprises finding multiple, respective transfer function components relating the first image formed over the first area on the end face of the multimode optical fiber to multiple second areas extending in different, respective locations over the side of the multimode optical fiber.

6. The method according to claim 1, wherein deriving the transfer function comprises generating the transfer function so as to decode the detected optical information regardless of changes in the transmission due to variations in a shape of the multimode optical fiber.

7. The method according to claim 1, wherein inputting the optical information comprises applying a pattern of light to the end face of the multimode optical fiber, and decoding the detected optical information comprises reconstructing the pattern by applying the transfer function to the light emitted from the side of the multimode optical fiber.

8. The method according to claim 1, wherein inputting the optical information comprises receiving light through the side of the multimode optical fiber, and decoding the detected optical information comprises outputting an image of an area radial to the multimode optical fiber responsively to the light received through the side of the multimode optical fiber.

9. The method according to claim 1, wherein at least the second area of the side of the optical fiber is jacketless.

10. Apparatus for transmitting information, comprising:
a multimode optical fiber, which includes a first area on an end face of the multimode optical fiber and a second area extending over a side of the multimode optical fiber;
an optical input assembly, which is configured to input optical information to the multimode optical fiber through one of the first and second areas;
a detector, which is configured to detect the optical information that is output from the other of the first and second areas; and
a processor, which is configured to derive a transfer function that relates a first image formed over the first area on the end face of a multimode optical fiber and a second image formed over the second area extending over the side of the multimode optical fiber, to receive the optical information detected by the detector, and to decode the detected optical information using the transfer function.

11. The apparatus according to claim 10, wherein the second image comprises a speckle pattern formed by Rayleigh scattering of coherent light within the optical fiber.

12. The apparatus according to claim 10, wherein the processor is configured to derive the transfer function by causing the optical input assembly to project multiple input images onto the first area, receiving from the detector respective output images of the second area responsively to the input images, and optimizing the transfer function so as to transform the input images into the respective output images.

13. The apparatus according to claim 12, wherein optimizing the transfer function comprises training a neural network to relate the respective output images to the input images.

14. The apparatus according to claim 10, wherein the processor is configured to derive multiple, respective transfer function components relating the first image formed over the first area on the end face of the multimode optical fiber to multiple second areas extending in different, respective locations over the side of the multimode optical fiber.

15. The apparatus according to claim 10, wherein the processor is configured to derive the transfer function so as to decode the detected optical information regardless of changes in the transmission due to variations in a shape of the multimode optical fiber.

16. The apparatus according to claim 10, wherein the optical input assembly is configured to apply a pattern of light to the end face of the multimode optical fiber, and the processor is configured to reconstruct the pattern by applying the transfer function to the light emitted from the side of the multimode optical fiber.

17. The apparatus according to claim 10, wherein the multimode optical fiber is configured to receive light through the side of the multimode optical fiber, and the processor is configured to decode the optical information that is received at the end face so as to output an image of an area radial to the multimode optical fiber responsively to the light received through the side of the multimode optical fiber.

18. The apparatus according to claim 10, wherein at least the second area of the side of the optical fiber is jacketless.
